(19) United States
(12) Patent Application Publication
(10) Pub. No.: US 2014/0345625 A1
(43) Pub. Date: Nov. 27, 2014

(54) DYNAMIC TRUNK LEANING SUPPORT

(71) Applicant: Mohammad ABDOLI-ERAMAKI, North York (CA)

(72) Inventor: Mohammad ABDOLI-ERAMAKI, North York (CA)

(21) Appl. No.: 14/287,530

(22) Filed: May 27, 2014

Related U.S. Application Data
(60) Provisional application No. 61/827,704, filed on May 27, 2013.

(51) Int. Cl. A61F 5/37

(52) U.S. Cl. CPC A61F 5/37
USPC 128/845

(57) ABSTRACT

A device is provided for supporting a portion of the upper body during forward lean. The device comprises a two pillars anchored to the ground or a stable platform. One or more linkage elements are connected between pillars. A support plate is slidably situated on the linkage elements approximately midway between the pillars. The support plate provides support to a user in the region of the user's breast plate. As the user leans forward, the lean forces are transferred through the linkage elements to the pillars, causing the pillars to flex, and thus provide support to the user. The height of the pillars and the stiffness of the flexible portion of the pillars are adjustable during the operation of the device.

### RELATED APPLICATIONS

This application claims priority from U.S. patent application 61/827,704, filed May 27, 2013. Priority is claimed to this earlier filed application and the contents of this earlier filed application is incorporated herein, in its entirety, by reference.

### FIELD

The present invention pertains to back support systems, in particular to a reaching back support system that provides back support during forward reach flexion through a three-dimensional range of motions.

### BACKGROUND

Standing in a forward flexed position is an awkward posture due to the flexion extension moment created by the trunk as it bends forward away from the centerline or neutral position. To hold this position the low back and hip extensor muscles must work continuously to counterbalance the moment composed of the weight of the upper body and external load, when applicable, times the length of the lever arm. Since the back extensors (erector spinae) work on a small lever arm an amplified tensile force is needed and as a result applies large compressive and shear forces through the lumbar spine. Stationary loading has been shown to increase the incidence of low back pain (Silverstein, Silverstein and Franklin, 1996), therefore lightening the biomechanical load on the lumbar spine should reduce the incidence of low back injuries. Once an injury has occurred, workers may be delayed in their return to work as they wait for sufficient healing and strengthening to take place so as their physical capacity matches the work demands.

Standing at work is a prevalent work posture (Tissot et al. 2005). In sitting, chair backrests have been shown to lower the muscular electromyography activity of the back extensors and abdominals (Makhsous, 2003) but this is not helpful when an individual leans forward to carryout a work task in front. An on-body personal lifting assist device (PLAD) has been shown to offset the spinal loads and reduces the electromyography of the erector spinae by 14%-21% and the compression and shear forces at L4/L5 by 13% to 15% (Abdoli et al. 2007, Lotz et al. 2007, Graham et al. 2008, Frost et al. 2008). PLAD is modeled on the concept of human muscle through the use of an elastic element that acts as an external muscle force generator but it has certain limitations associated with being an on-body device and appears best suited for dynamic work such as manual handling tasks with large vertical lifting. Alternate work postures include sitting or standing upright in a neutral position. Standing compared to sitting produces lower compressive forces through the lumbar spine (Callaghan & McGill, 2001), Psychophysically, however, the preference and perceived effort is mixed. Yates and Karwowski, (1992) found subjects perceived sitting to be harder and the authors attributed the difference to change in lumbar curvature. Kim et al., (2004) also found a higher perceived load in sitting but this was only for smaller subjects. Johnson and Nussbaum (2003), found that perceived effort was higher in a standing waist bend posture possibly due to more stability gained from leaning onto the fixture. An early study by Aaras et al. (1988) also showed that a seated posture was usually preferred despite increased load on the shoulder muscles and attributed to improved precision, stability, increased mobility and less load on legs and feet, less energy expenditure. Standing upright with the flexion extension moment neutral is ideal, however, this is not always possible and a forward flexed posture is needed when a work surface is below the elbow height or extreme reach beyond the length of the extended arm is needed. These two factors are unique to individuals and cannot always be accommodated by traditional ergonomic strategies.

Low back pain is the most common musculoskeletal complaint of workers with annual costs estimated at $12 Billion in 2002 in Canada (WorksafeBC, 2003) and $90 Billion in 1998 in the United States (Luo et al. 2004). Standing work has been identified as a risk factor for lower back pain as a result of the cumulative spinal loading and physiological work demands that result from the flexion moment that is created when the trunk leans forward from the upright neutral position. With as little as 10 degrees of forward lean the compressive loads through the lower lumber discs doubles (Takahashi et al., 2006). A recent population survey in Quebec showed that 58% of workers reported standing at work of which less than less 20% report that they can alternate position by sitting or walking (Tissot et al., 2005). Keyserling (1992), reported 89% of 335 surveyed manufacturing and warehouse jobs involved mild trunk flexion of less than 20 degrees. The degree of forward inclination depends upon individual anthropometry, design of workstation and nature of the work. The amount of forward leaning can be minimized by mechanical strategies that adjust workstation to fit the individual but this is not always practical, safe and can be costly.

Low back pain is disruptive to ongoing business operations not only in terms of lost work time but also lost productivity when workers are unable to carry out their full work duties. Stewart et al. (2003) reported that in a 2 week period, 13% of the total workforce in the United States experienced a loss in productive time, the majority of which occurred while at work, due to a pain complaint. Low back pain accounted for 3.2% of these pain complaints. Workplace injury prevention is therefore an important part of cost management by employers. Takashi et al (2006) reported a mechanical load increase of approximately 360% of the intervertebral disc in a forward leaning position of 30 degrees trunk flexion. This extra effort that the muscles must do to over time results in musculoskeletal disorders (MSDs) and contributes to injuries and lost time from work. Nearly hold of all claims registered with the WSIB relate to MSDs. During 1999-2003, there were a total of 210,663 lost-time injuries in Ontario as a result of MSDs. Direct costs due to MSDs for the period 1996-2002 totaled more than an estimated $2.2 billion. Conservatively, the sum of the direct and indirect costs due to MSDs is estimated to be approximately $10 billion (Prevention Strategy for MSDs in Ontario, 2005).

An ergonomic device that safely lowers compressive loads may be effective in addressing the above-noted problems.

### SUMMARY

According to an aspect a device for supporting a portion of the upper body during forward lean is provided. The device can comprise:

at least two pillars, a portion of each pillar being flexible;

a linkage element having two ends, each end operably attached to the flexible portion of each said pillar;

a support plate slidably situated on said linkage element, said support plate providing support to a user in the region of the user's breast plate, said support plate receiving lean forces during periods of forward lean and delivering said lean forces to said flexible portion of said pillars via said linkage element, thereby transferring at least a portion of the upper body weight of the user to said flexible portions.

The pillars can be height adjustable. The stiffness of the flexible portion can be adjustable. The pillars can be placed symmetrically about the support plate when the device is operated. Each of the pillars can include three telescoping shafts, one of the shafts being flexible and forming the flexible portion of each of the pillars.

### BRIEF DESCRIPTION OF THE DRAWINGS

Implementations will now be described, by way of example only, with reference to the attached Figures, wherein:

FIG. 1 is a perspective view of the DTLS device in accordance with an implementation;

FIG. 2 is a perspective view of a support plate in accordance with an implementation;

FIG. 3 is a perspective view of a support plate in accordance with an implementation;

FIG. 4 is a sectional view of a pillar showing parts of the linkage elements in accordance with an implementation;

FIG. 5A is a is a side sectional view of the support plate in accordance with an implementation;

FIG. 5B is a schematic representation of suitable support plate shapes plate in accordance with an implementation;

FIG. 6 is a sectional view of a pillar in accordance with an implementation;

FIG. 7 is a sectional view of a flexible rod in accordance with an implementation;

FIG. 8 is a perspective view of a configuration of the DTLS device on a bucket in accordance with an implementation; and

FIG. 9 is a perspective view of the DTLS device where the user is sitting in accordance with an implementation.

### DESCRIPTION OF ILLUSTRATIVE EMBODIMENTS

The dynamic trunk leaning support (DTLS) device is a mechanical device that reduces biomechanical loading and physiological work of the lower back by continuously supporting the trunk during forward leaning. It has been determined that supporting a portion of the upper body weight reduces the flexion moment thus reducing the muscular work necessary to counterbalance the moment and resulting spinal load. The device is intended to reduce the incidence of lower back injury, promote an early return to work following a lower back injury and increase stability in standing. The DTLS device described herein is designed to support a portion of the body weight through the bony ribcage while allowing full axial movement.

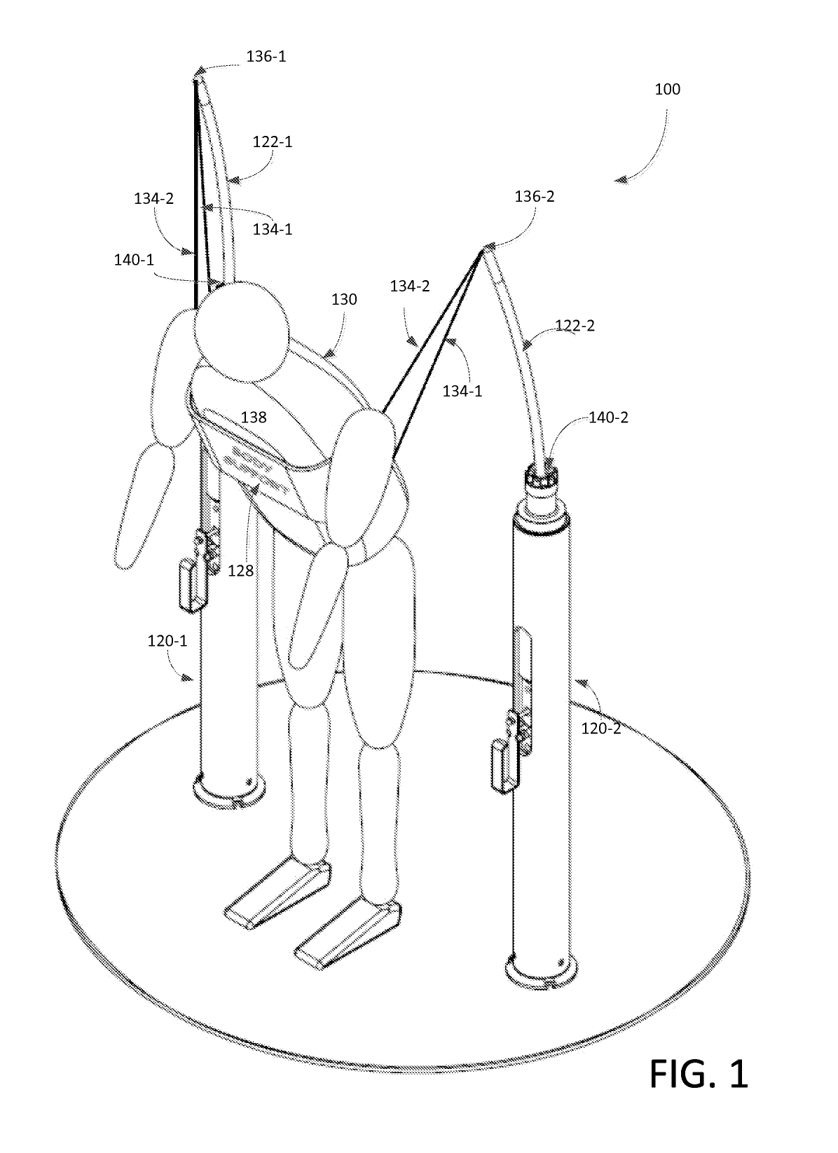

Referring now to FIG. 1, shown is an implementation of the DTLS device 100. The DTLS device 100 generally comprises vertical pillars 120-1 and 120-2. Collectively, pillars 120-1 and 120-2 are referred to as pillars 120, and generically as pillar 120. In the implementation shown, two pillars 120 are provided, placed symmetrically about a support plate 128 and accordingly user 130. While represented as two pillars, the DTLS device 100 can include a plurality of pillars greater than two, with the arrangement of the pillars generally being symmetrical about a support plate 128 and thus user 130. For example, the plurality of pillars may be arranged in the form of a linear or semicircular array, comprising 4 or more individual pillars. By maintain a symmetric arrangement of pillars, appropriate support is provided for the torso of user 130 that engages in asymmetrical movement such as lateral or side bends or twists of their torso in addition to leaning directly forward. The pillars 120 can be attached to the ceiling, to the walls, to the floor, to a movable platform where the user stands, or any other stable surface.

The DTLS device 100 also includes two linkage elements 134-1 and 134-2. Collectively, linkage elements 134-1 and 134-2 are referred to as linkage elements 134, and generically as linkage element 134. This nomenclature is used elsewhere herein. Each linkage element 134 has one end attached to pillar 120-1 at the attachment point 136-1, and the other end attached to the other pillar 120-2 at attachment point 136-2 as indicated in FIG. 1. Linkage elements 134 are, in one implementation, wires or cables made out of metallic, plastic or other suitable material with sufficient flexibility and thickness so as to provide the appropriate shape and strength to transfer forces generated by leaning forward, or lean forces, from the user 130 to the pillars 120 by engaging the user through the support plate 128. In variations, the linkage elements can be flat or round cords, straps or other elements that will now occur to a person of skill. To attach a linkage element 134 to a pillar 120, a variety of fastening mechanisms can be implemented. For example, the attachment points 136 can be configured with suitable fasteners that releasably retain an end of the linkage element 134. Alternatively, the linkage elements 134 can be affixed directly to pillars 120 using suitable fasteners or engagement anchors. In the implementation shown, two linkage elements 134 are provided. In variations, one, three or more linkage elements can be used.

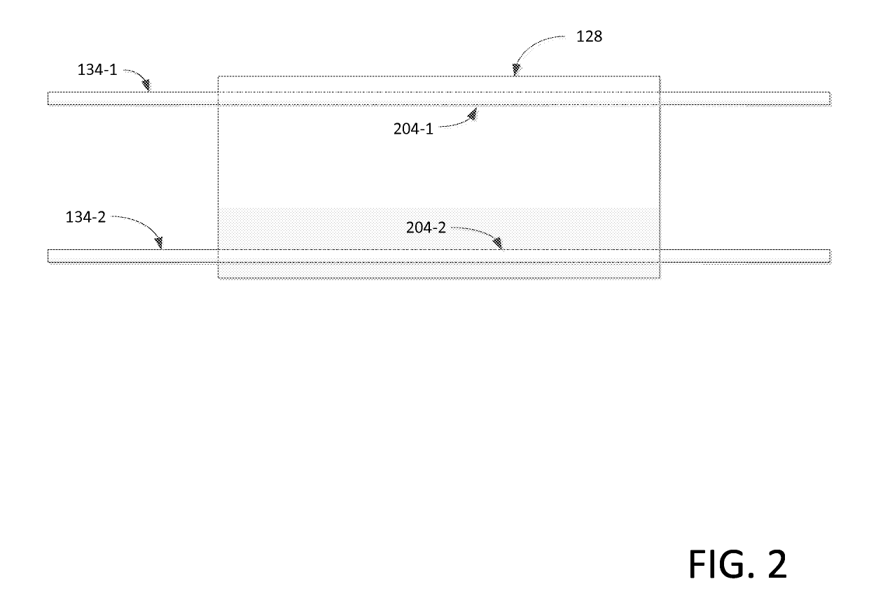

Continuing with FIG. 1, towards the middle of the linkage elements 134, a support plate 128 is slidably situated. As will be described in greater detail below, a user 130 of the DTLS device 10 will generally stand or sit in front and approximately equidistant from each pillar 120 as shown in FIG. 1, wherein the support plate 128 is configured to align to the user at the level of the bony ribcage 138. Turning now to FIG. 2, in accordance with an implementation, the linkage elements 134 can slidably pass through channels 204-1 and 204-2 formed within the support plate 128. Collectively, channels 204-1 and 204-2 are referred to as channels 204, and generically as channel 204. The channels 204 can be formed through various mechanisms that will now occur to a person of skill, such as material sawn onto the pads of support plate 128, or cavities formed within the support plate 128. Although in FIG. 2, the channels 204 are indicated towards the upper and lower edges of the support plate 128, in variations, they can be located anywhere on the support plate 128, as long as they are generally aligned with the linkage elements 134 so as to allow the support plate 128 to slide along the linkage elements 134. Accordingly, the support plate 128 can slide to the left or to the right over the linkage elements 134, as the user engages in asymmetrical movement such as lateral or side bends or twists of their torso in addition to leaning forward. This in turn allows for a more balanced support of the user 130 when user 130 is engaged in lateral or side movement by allowing the support plate to remain in a constant position with respect to the user 130's torso.

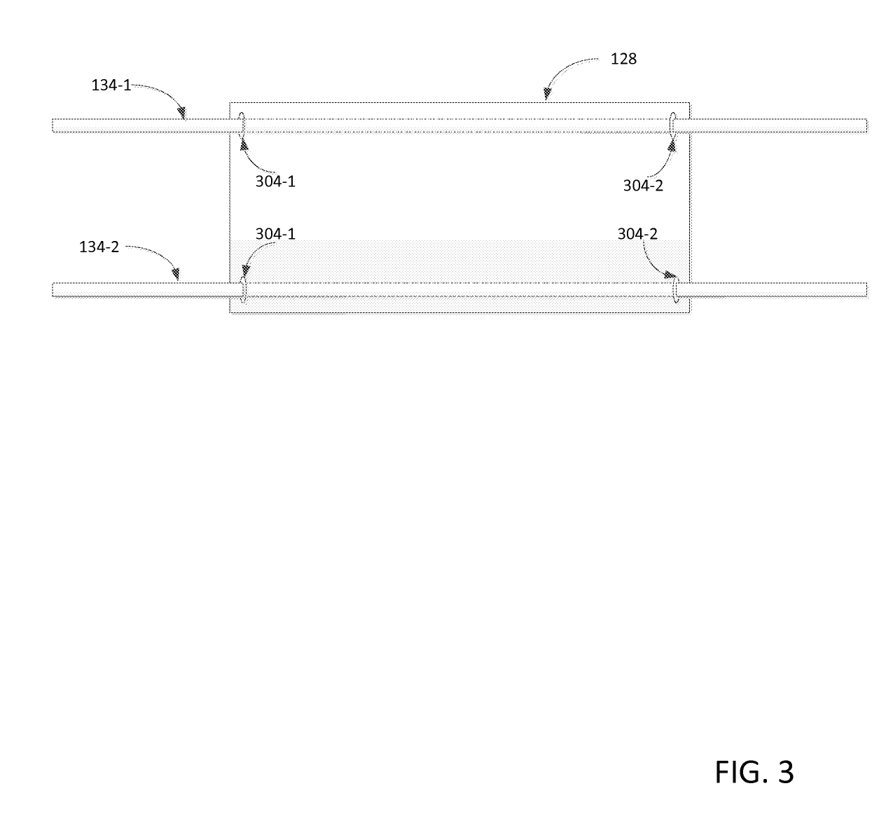

Other mechanisms for slidably situating a support plate 138 on linkage elements 134 will now occur to a person of skill in the art. For example, as shown in FIG. 3, each linkage element 134 could pass through a pair of holes 304-1 and 304-2 through the support plate and thus a portion of the linkage element passing along the opposite face of the support plate from which it enters the holes as shown in FIG. 3. Other variations of slidably situating a support plate 128 on linkage elements 134 will now occur to a person of skill.

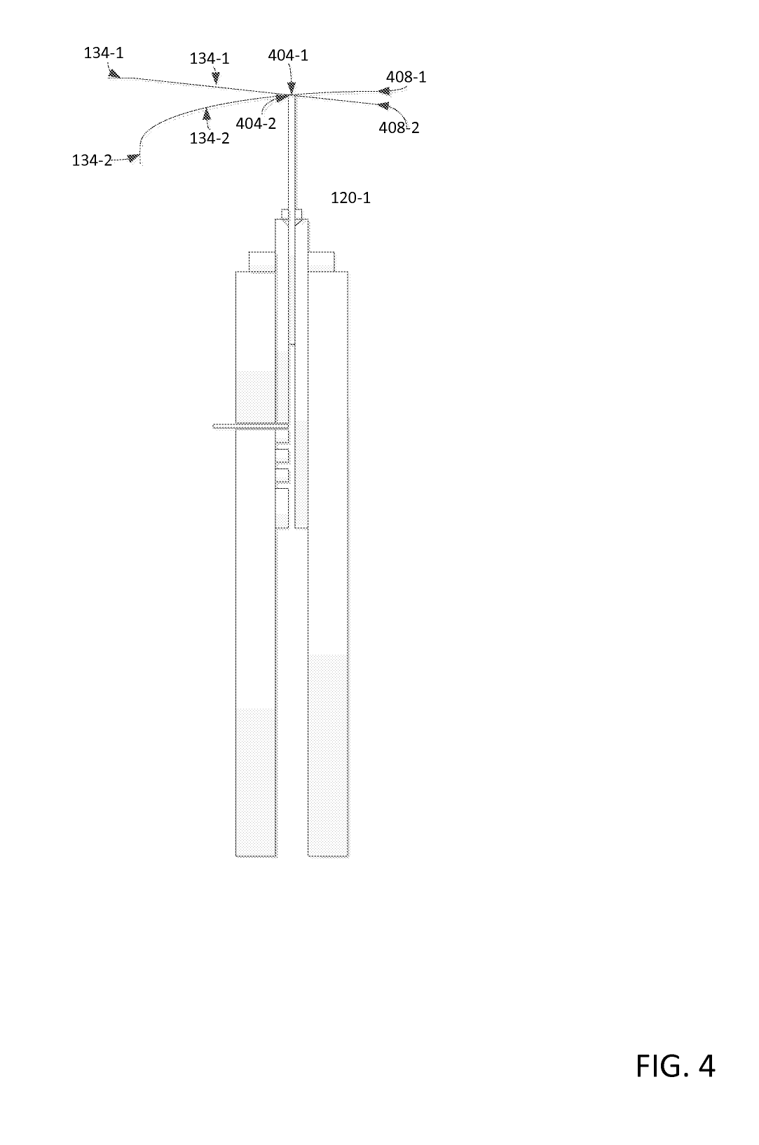

The length of the linkage elements 134 that is operably available to transfer lean forces can be dynamically adjusted during the use of the device 100. For example, referring to FIG. 4, linkage elements 134 can be attached to a pillar 120 at linkage locations 404-1 and 404-2 on each linkage element 134 respectively. Collectively, linkage locations 404-1 and 404-2 are referred to as linkage locations 404, and generically as linkage locations 404. The linkage points 404 are further towards the middle of the linkage elements 134 than their endpoints 408-1 and 408-2. Accordingly, the operable length of the linkage elements is shortened since the portion of the linkage elements 134 that are between linkage location 404 and endpoints 408 are not available as operable length of the linkage elements 134. FIG. 4 only shows a portion of the linkage elements 134, the portions beyond the dotted lines 412-1 and 412-2 not being shown. To attach a linkage element 134 to a pillar 120 at a linkage location 404, a variety of fastening mechanisms can be implemented. For example, the attachment points 136, shown in FIG. 1, can be configured with suitable fasteners that releasably retain the linkage elements 134 at linkage locations 404. Shortening the operable length of the linkage elements allows the adjustment of the device 100 for user preferences and work conditions, by providing more or less angle of flexion prior to the device providing support, for example. In variations, multiple linkage locations can be provided along the length of the linkage elements 134, allowing the operable length of the linkage elements to be dynamically adjusted to various different operable lengths.

Various shortening mechanisms will now occur to a person of skill for dynamically changing the linkage location at which a linkage element is attached to a pillar. For example, a shortening mechanism having a spool attached to attachment points 136, or other locations on the pillar can be used. Accordingly, the operable length of the linkage elements 134 can be adjusted by using the spool mechanism. In variations, the tightening or shortening mechanism can be located on the support plate 128.

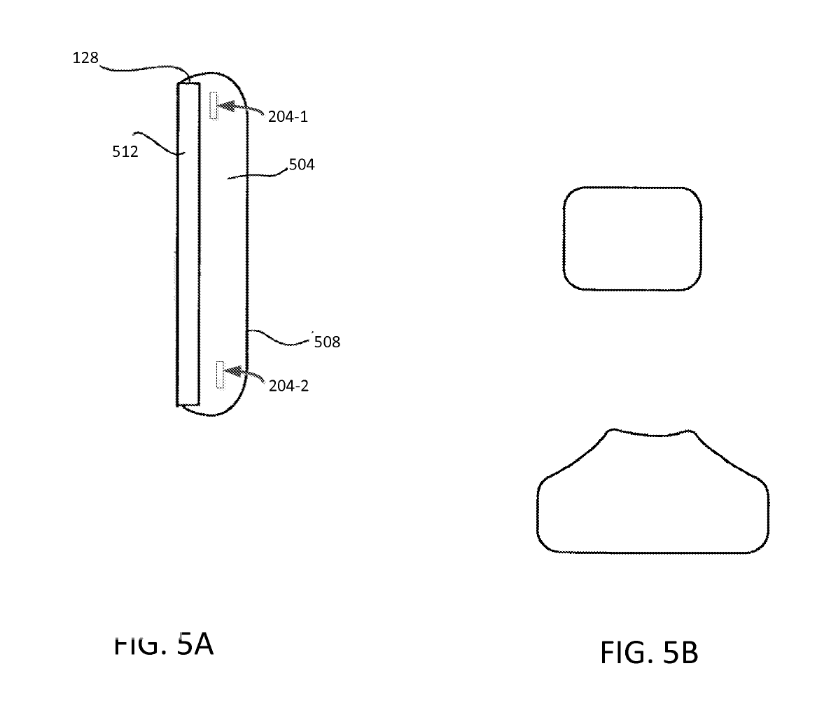

Support plate 128 is constructed using materials generally known in the art. In general, as shown in FIG. 5A, at least one layer of suitable padding, such as foam 504, is provided, over which a suitable material 508 (e.g. fabric, plasticized fabric, vinyl, etc.) is placed. Optionally, a base 512 for rigidity comprised of a metal, plastic or composite can also be included. The support plate 128 is relatively small and generally shaped to optimally distribute the body weight forces over an anatomically appropriate area of the bony ribcage. The support plate 128 is sized to allow freedom of movement of the shoulder girdle and upper extremities, as well as avoiding compression of the soft tissue of the abdomen, and female breasts. Exemplary shapes for the support plate are shown in FIG. 5B.

In some implementations, the attachment point at which the linkage elements are attached to a pillar 120 are aligned with the shoulder height of the user 130 when the user 130 is in neutral position such as standing, kneeling or sitting without any lean. Such an alignment allows for appropriate provision of support for full range of lean. To accommodate users of different height, the pillars 120 can be provided with a height adjustment mechanism. Height adjustability can be provided through any number of mechanisms. For example, height adjustability may be provided through the use of telescoping members. Alternative height adjustment mechanisms will now occur to a person of skill.

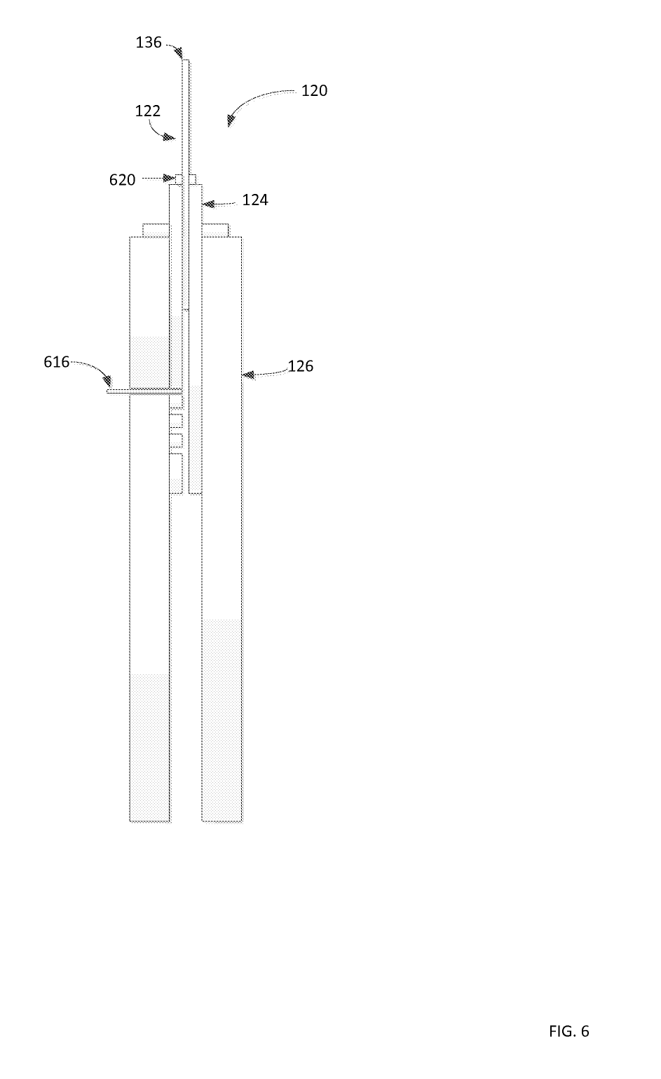

Referring with FIG. 6, each pillar 120 is provided as a telescoping shaft comprising flexible shaft 122, and an intermediary shaft 124 within which the flexible shaft 122 is slidable in a telescoping arrangement. Each pillar 120 further includes an anchor shaft 126 within which the intermediary shaft 124 is slidable in a telescoping arrangement. As shown in FIG. 6, the intermediary shaft 124 is configured with an internal diameter that is greater than the outside diameter of the flexible shaft 122, thereby allowing the flexible shaft 122 to telescope within the intermediary shaft 124. As a person of skill will appreciate, the opposite arrangement is also possible whereby the diameters of the flexible and intermediary shafts are such that the flexible shaft 122 is configured to telescope upon the intermediary shaft 124. Also as shown, the anchor shaft 126 is configured with an internal diameter that is greater than the outside diameter of the intermediary shaft 124, thereby allowing the intermediary shaft 124 to telescope within the anchor shaft 126. As a person of skill will appreciate, the opposite arrangement is also possible whereby the diameters of the anchor and intermediary shafts are such that the intermediary shaft 124 is configured to telescope upon the anchor shaft 126. In further variations, the flexible shaft can be the intermediary shaft 124.

To set the telescoping support shaft at a particular height, two locking mechanisms 616 and 620 are provided. Locking mechanisms suitable for locking the telescoping shafts can take many different forms as it will now occur to a person of skill in the art. For example, a quick adjust mechanism such as a spring-biased pin for cooperating with a plurality of holes in at least one of the shafts can be provided as indicated at 616. Alternatively, a set screw mechanism can be provided to set a particular height (not shown). In a further variation, the height of the pillar 120 can be governed by a threaded engagement between two shafts. In such an arrangement, the first and second shafts are provided with respective cooperating threads. Rotation of the first shaft relative to the second shaft has the effect of lengthening or shortening the pillar 120, effectively raising or lowering the attachment point 136. Further, alternate arrangements for adjusting the height of the pillar 120 may be used that will now occur to a person of skill. For example, a collar can be placed at the top end of a telescoping shaft, as indicated at 620, within which another shaft is slidably placed. The collar can be used to hold the sliding shaft, in this case flexible shaft 122 at a fixed position in relation to the shaft within which it telescopes, in this case the intermediary shaft 124. The collar can utilize a screw mechanism (not shown) to engage the intermediary shaft 124 and can cause the intermediary shaft's upper opening through which the flexible shaft 122 is slidably inserted, to be narrowed as the collar is tightened by turning. Thus, the flexible shaft 122 is held in place relative to the intermediary shaft 124 by friction. Alternatively, the collar can cause the opening of the intermediary shaft 124 to widen by loosening the collar by turning it counter-clockwise, allowing the flexible shaft 122 to once again slidably move within the intermediary shaft 124.

Referring back to FIG. 1, when a user 130 leans directly forward, the forward lean exerts forces on the support plate 128 in the direction of the lean. The support plate, in turn, transmits the lean forces to the pillars 120 by pulling the flexible shafts 122 at attachment points 136 through the linkage elements 134. Accordingly, the forward lean upon the support plate 128 imparts a given amount of flexion stress upon the flexible shafts 122, operably associated with the linkage points of the linkage elements 134. The result of the flexion stress on the flexible shafts 122 is the flexing of the flexible shafts 122 as shown in FIG. 1. The flexing of the flexible shafts 122, in turn, generate support forces, generally in the opposite direction of the lean forces, thus providing support for the user 130. In some implementations, where the linkages 134 are stretchable such as through the use of flexible materials or spring elements, the linkage elements 134 may also stretch in response to the lean, providing additional support forces in the form of linkage contraction. Adjusting the operable length of the linkage may adjust the amount of support forces provided in the form of contraction.

When the forward lean includes a lateral move such lateral or side bends or twists of the torso in addition to leaning forward, the support plate 128 can slide to the left or to the right over the linkage elements 134. This in turn allows the support plate to remain in a constant position with respect to the user 130's torso. However, the operable length of the linkage elements between the support plate and the two pillars is no longer the same due to the slide of the support plate 128 with respect to the linkage elements 134. Accordingly, when the user leans asymmetrically to their right, the support plate 128 slides along linkage elements 138 to the user's right as well, and thus is no longer centered along the length of the linkage elements 134. As a result, the operable length of the linkage elements between the two pillars is also asymmetrical, the operable length to the pillar 120 to the right of the user 130 being shorter than the operable length to the pillar 120 to the left of the user 130. Based on the asymmetrical bend and the resulting asymmetrical position of the support plate 128, each flexible pillar experiences a different amount of flex, and thus the two pillars provide asymmetrical support forces for the user 130, appropriately countering the lean forces of the user 130.

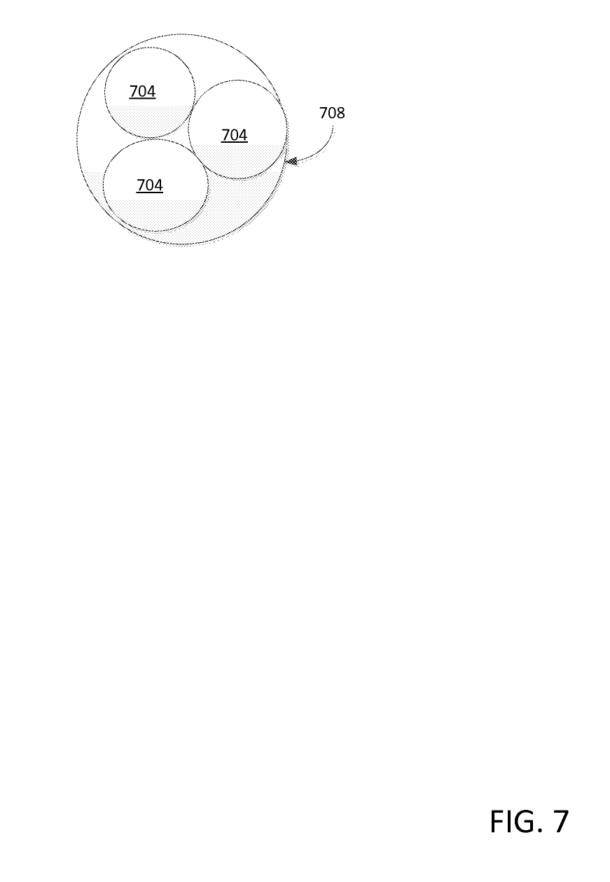

The flexible shafts 122 are made of any materials that can flex when a pulling force is applied at one end, perpendicular to its longitudinal axis, while the other end is held stationary, causing it to bend as shown in FIG. 1. In one implementation, the flexible shafts 122 are provided as rods made from flexible materials such as metals, plastics, carbon fiber, and others that will now occur to a person of skill in the art. In variations, the flexible rods can be made of composite components and, as shown in FIG. 7, include several flexible rods 704 that are encased within a flexible tube 708. Different rods 704 can be made of different materials and compositions. The type and composition of the flexible shafts 122 determine their stiffness and the type and materials and composition can be chosen accordingly to provide the desired stiffness range. Other materials, compositions and arrangements for constructing flexible rods will now occur to a person of skill.

The extent of pull on each of the flexible shafts 122 is dependent upon the extent of lean, and the direction of lean relative to the neutral position. As one will appreciate, in a forward lean directed towards the right-hand side, flex stresses are delivered via the linkage elements in a disproportionately greater manner to the left-hand side pillar 120, and vice versa. Motion of the user 130 through the allowable range of forward lean has the effect of dynamically transferring to the flexible shafts 122 a portion of the upper body weight. Support of the upper body is achieved by way of redirecting the load through the linkage elements into the flexible shafts 122 in the form of flex stresses, thereby supporting the forward lean throughout based on the stiffness of the flexible shafts 122.

The stiffness of the flexible shafts 122, and thus the resistance they offer to lean forces can be dynamically adjusted during the operation of the DTLS device 100 to suit anthropometric differences as well as user preference and differing user body sizes and weights. A flexible shaft 122 is any shaft that can flex when a pulling force is applied at one end, perpendicular to its longitudinal axis, while the other end is held stationary, causing the shaft to bend as shown in FIG. 1. The amount of flex exhibited against a given pull force determines the stiffness and thus the resistance a flexible shaft provides against a pull, and is thus partly determinative of the support provided to a user during a forward lean. The stiffness of a flexible shaft can be adjusted, in part, by adjusting the axial length, referred to herein as the flex distance, of the flexible shaft available for flexing. In the present implementation the axial length, or the flex distance, relevant to the stiffness of a flexible shaft is the distance between the attachment point 136, where the linkage elements are attached to the flexible shaft 122 and the insertion point 140 at which point the flexible shaft 122 enters the intermediary shaft 124 (shown as 140-1 for shaft 122-1 and 140-2 for shaft 122-2). Accordingly, by sliding more of the flexible shaft 122 out of the intermediary shaft 124 through telescoping movement, thus increasing the flex distance, the flexibility of the flexible shaft 122 can be increased. Conversely, by sliding more of the flexible shaft 122 into the intermediary shaft 124, thus decreasing the flex distance, the stiffness of a flexible shaft 122 can be decreased.

An alternative mechanism for changing stiffness is to change the location of the attachment point 136, by for example moving it below the upper tip of the flexible shaft, and closer to the insertion point 140, thereby effectively reducing the flex distance. The attachment point 136 is effectively moved by moving the point at which the linkage elements 134 are attached to the flexible shaft 122.

As discussed above, in some implementations, the attachment point 136 is kept at shoulder height. Accordingly, as the stiffness of flexible shaft 122 is adjusted either by telescoping flexible shaft 122 in or out of intermediate shaft 124 or by moving the point at which the linkage elements 134 are attached to the flexible shaft, the attachment point 136 can be kept at shoulder height by telescoping the intermediary shaft 124 with respect to the attachment shaft 126, as appropriate.

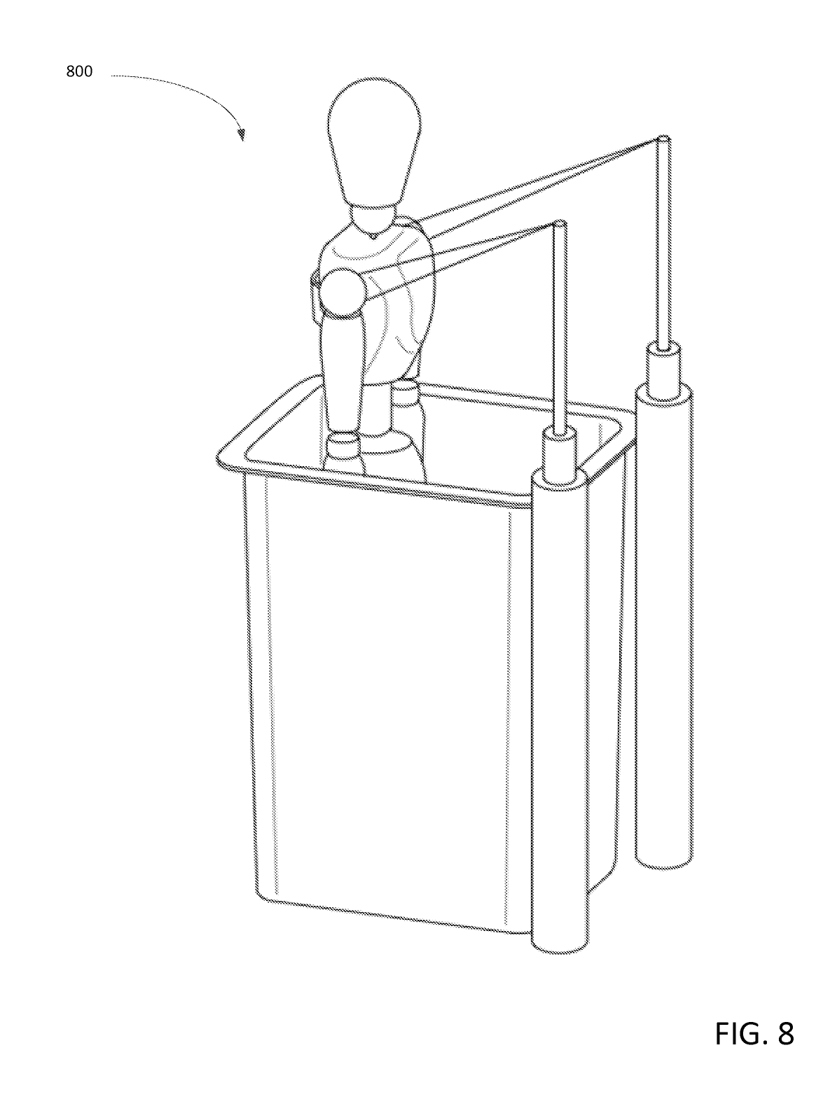

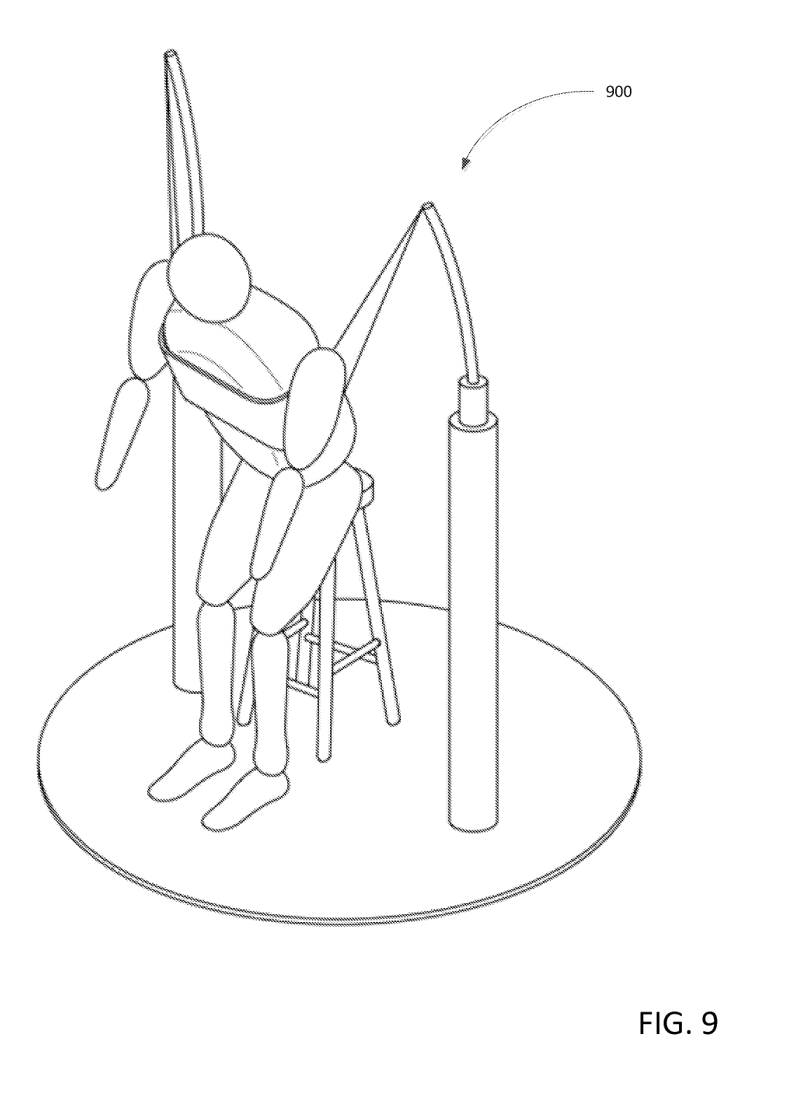

The DTLS device 100 is suitable for use as a stand-alone device, or in combination with a platform wherein pillars 120 are anchored to a support platform upon which the user stands. To facilitate transport of the DTLS device 100, for example where used in environments where the user must move from one location to another, the support platform may be provided with wheels. The wheels are configured to retract or are otherwise arranged for usage only during movement of the device, such as when the stand-alone device is tilted for movement from one location to another. In alternative implementations, the pillars can be attached to corners of a bucket, as indicated in FIG. 8 at 800. In alternative implementations a user 130 can be in a sitting position and the height of the pillars adjusted to accommodate the user's shoulder height as indicated in FIG. 9 at 900.

It may also be advantageous to configure the stand-alone or bucket versions of the DTLS device with a fold-away or retractability feature. In particular, in the event the DTLS device presents an obstruction or impediment to work when forward leaning is not necessary, the ability to retract or fold-away the DTLS device 100 would be desirable.

In use, the DTLS device can provide (1) continuous partial support through a range of motion, (2) three dimensional trunk movement, (3) load transfer over the most stable part of the anterior rib cage, (4) no interference with arm mobility, (5) no compression in the thoracic outlet area.

In one study that analyzed single plane forward flexion through a trunk angle of 0 to 50°, the DTLS device was able to reduce the compression forces at the L4-L5 joint between 10 to 70% at 10 and 50 degrees respectively (see FIG. 11). In a further study, analyzing single plane forward flexion through a trunk angle of 0 to 40°, the DTLS device was able to reduce peak EMG (L4-L5 joint) between 5 to 55% at 10 to 40 degrees respectively.

The DTLS device may find application in a range of areas, including (1) industry, healthcare and the service sector, (2) orthopaedic rehabilitation, and (3) home support for the disabled and elderly.

The DTLS device may also be used as a prescriptive clinical work brace for orthopaedic rehabilitation for injured workers with lower back injuries. It could be used in workplaces to facilitate a safe return to work following a significant low back injury such as disc protrusion, spinal fracture or instability. Further, the DTLS device could be a prescriptive leaning device for elderly individuals at home who experience problems with balance and/or generalized weakness. It could be used as a postural support while carrying out activities of daily living, for example while brushing teeth or combing hair.

The DTLS device may be marketed as a preventative ergonomic device for use in the workplace, particularly in the manufacturing, healthcare, waste management and the service sector where a significant portion of work is carried out in forward leaning trunk postures. The device reduces a worker's exposure to compressive and shear forces through the lumbar spine that eventually translate into lower back discomfort, productivity loss and injury claims. Other benefits from the use of the DTLS device include increased core stability with an offbalancing reach, reduction in the physiological cost of work resulting from lessened postural muscular workload, promotion of a suitable spinal posture rather than a forward slump, and limitation of rotational movements during forward flex.

The DTLS device may be used as a clinical assistive device used in the early postoperative stage following a lower back injury and/or surgery or at a later recovery stage to protect bones and joints from compressive and shear joint forces and contractile soft tissue from excessive muscular tension following repair of a spinal fracture or disc protrusion. The DTLS device could be adapted to provide an adjustable level of support that can be changed according to the stage of healing and tissue tolerance and adjustable limits for range of motion in three dimensions. For example, a surgeon could prescribe maximum of 30 degrees forward flexed trunk with no rotation and 80% support. In the later stage of recovery, typical functional restoration programs are directed to progressively increase tissue loading until there is adequate tolerance and reduced fear of pain or re-injury. The DTLS device may serve as an adjunct to existing rehabilitation interventions. The DTLS device may promote an earlier return to pre injury or modified work by simply lowering the spinal loads and muscular workload of the job and reducing the likelihood of an early onset of local back muscle fatigue.

The DTLS device may also be used to assist seniors with diminished balance, generalized weakness, or shortened reach distance. Walking aids used for balance require the use of both hands and therefore are not available to carry out simple tasks. Given the DTLS device does not limit arm use, it could be used as a postural support while carrying out activities of daily living, such as brushing teeth or combing hair.

Postural supports are generally rigid in nature and can impede normal work movement patterns and be uncomfortable to wear or use. The DTLS device is based on a biomechanical principle using leverage and support to lower physiological demands. It is based on dynamic splinting providing partial support, thereby limiting the risk that the spinal joints and discs are kept in a static position. Physiological movement is active assisted thereby preventing muscle atrophy from disuse and work specific deconditioning.

It will be appreciated that, although implementations have been described and illustrated in detail, various modifications and changes may be made. While several implementations are described above, some of the features described above can be modified, replaced or even omitted. All such alternatives and modifications are believed to be within the scope of the invention and are covered by the claims appended hereto.

1. A device for supporting a portion of the upper body during forward lean, said device comprising:
at least two pillars, a portion of each pillar being flexible;
a linkage element having two ends, each end operably attached to each said pillar;
a support plate slidably situated on said linkage element, said support plate providing support to a user in the region of the user's breast plate, said support plate receiving lean forces during periods of forward lean and delivering said lean forces to said flexible portion of said pillars via said linkage element, thereby transferring at least a portion of the upper body weight of the user to said flexible portions.

2. The device according to claim 1, wherein said pillars are height adjustable.

3. The device according to claim 1, wherein a stiffness of said flexible portion is adjustable.

4. The device according to claim 1, wherein said pillars are placed symmetrically about said support plate when said device is in operation.

5. The device according to claim 3, wherein an operable height of said flexible portion is adjustable and said stiffness of said flexible portion is determined based on said operable height.

6. The device according to claim 5, wherein each said pillar includes at least two telescoping shafts, one of said shafts of each pillar being flexible and forming said flexible portion of each said pillar.

7. The device according to claim 6 wherein the height of said flexible portion is adjusted by telescoping said flexible shaft with respect to the second shaft.

8. The device according to claim 5, wherein each said pillar includes at least a top, a middle and a bottom telescoping shaft, said top shaft of each pillar being flexible and forming said flexible portion of each said pillar.

9. The device according to claim 8 wherein said operable height of said flexible portion is adjusted by telescoping said flexible shaft with respect to the middle shaft of each said pillar.

10. The device according to claim 8 wherein a height of each said pillar can be adjusted by telescoping said middle shaft with respect to said bottom shaft of each said pillar.

11. The device according to claim 5, wherein each said pillar includes at least a top, a middle and a bottom telescoping shaft, said middle shaft of each pillar being flexible and forming said flexible portion of each said pillar.

12. The device according to claim 11 wherein said operable height of said flexible portion is adjusted by telescoping said flexible shaft with respect to at least one of said top shaft and said bottom shaft of each said pillar.

13. The device according to claim 1 wherein said pillars form a portion of a bucket for receiving a user.

14. The device according to claim 1 wherein at least a portion of said linkage element passes through one of: a channel defined within the support element;
and at least one opening defined within the support element.

15. The device according to claim 1 wherein said linkage element is stretchable.

16. The device according to claim 1 wherein each said end of said linkage element can be removably attached to each said pillar.

17. The device according to claim 1 wherein said linkage element includes a shortening mechanism for adjusting an operable length of said linkage element.

18. The device according to claim 17 wherein said linkage elements, in accordance with said shortening mechanism includes multiple attachment points for attaching said endpoints to said pillars, thereby allowing an alteration of said operable length of said linkage element.

19. The device according to claim 1 wherein said shortening mechanism is a spool for shortening said operable length of said linkage element.

20. The device according to claim 1 wherein each said flexible portion of each said pillar is composed of several flexible rods encased within a flexible tube.
